(19) United States
(12) Patent Application Publication
(10) Pub. No.: US 2007/0207483 A1
(43) Pub. Date: Sep. 6, 2007

(54) BUFFERS FOR DETECTION OF mRNA SEPARATED IN A MICROFLUIDIC DEVICE

(75) Inventor: Luis Ugozzoli, San Raphael, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/676,900

(22) Filed: Feb. 20, 2007

Related U.S. Application Data
(60) Provisional application No. 60/779,312, filed on Mar. 2, 2006.

(51) Int. Cl. C12Q 1/68; C12P 19/34

(52) U.S. Cl. 435/6; 435/91.2

(57) ABSTRACT

The present invention provides methods and kits using low conductivity buffers for the detection of mRNA from a cell lysate separated in a microfluidic device. The buffers do not affect the integrity of the cell nucleus, thereby allowing for generation of a cell lysate that can be directly used as a template in reverse transcriptase (RT)-polymerase chain reaction (PCR) amplification reactions without contaminating genomic DNA.

### CROSS-REFERENCE TO RELATED APPLICATIONS

This application claims the benefit of U.S. Provisional Application No. 60/779,312, filed on Mar. 2, 2006, the entire disclosure of which is hereby incorporated herein by reference.

### BACKGROUND OF THE INVENTION

Presently available buffers for preparation of mRNA from cell lysate disrupt the cell nucleus and are of a high conductivity (see, for example, U.S. Patent Publication 2005/0053952). High conductivity renders present buffers incompatible for use in a microfluidic device or in microfluidic procedures that utilize a combination of hydrodynamic and electrokinetic flow, including selective ion extraction (SIE). Further, disruption of the cell nucleus results in contaminating genomic DNA in the cell lysate.

The present invention addresses these shortcomings by providing buffers with sufficiently low conductivity to be suitable for use in microfluidic procedures and that do not disrupt the integrity of the cell nucleus.

### BRIEF SUMMARY OF THE INVENTION

The present invention provides methods and kits using low conductivity buffers for the production of a cell lysate that can be directly used as a template for the detection of mRNA without contaminating genomic DNA.

Accordingly, in a first aspect, the invention provides methods for detecting mRNA in a cell. In some embodiments, the method comprises:
a) contacting a cell with a buffer, thereby generating a cell lysate comprising an intact cell nucleus, wherein the buffer has a conductivity of 10 mS/cm or less;
b) separating the cell nucleus from the lysate, thereby producing a supernatant containing the mRNA;
c) removing mRNA in the supernatant from inhibitors of reverse transcriptase and inhibitors of DNA polymerase via microfluidic separation; and
d) amplifying at least one mRNA sequence with reverse-transcriptase polymerase chain reaction (RT-PCR), whereby the mRNA in the cell is detected.

In some embodiments, microfluidic separation comprises selective ion extraction (SIE).

In some embodiments, the step of separating the cell nucleus from the lysate is carried out by centrifugation.

In carrying out the present methods, genomic DNA (gDNA) is preferably not detectably amplified.

In another aspect, the invention provides kits comprising:
i) a substrate comprising microfluidic channels; and
ii) a buffer having a conductivity of 10 mS/cm or less.

In some embodiments, the substrate is suitable for use in selective ion extraction (SIE).

With regard to embodiments of the methods and kits, the buffer can have a conductivity of about 9, 8, 7, 6, 5, 4, 3, 2, 1 mS/cm or any value between about 0.5-10 mS/cm. In some embodiments, the buffer has a conductivity of 5 mS/cm or less. In some embodiments, the buffer has a conductivity of 2 mS/cm or less.

In some embodiments, the buffer comprises the following components at the following concentrations:
up to 50 mM sodium chloride, for example, 10, 20, 30, 40, 50 mM, or 5-20 mM NaCl;
up to 50 mM biological buffering agent, pH 6.0-8.3, for example 10, 20, 30, 40, 50 mM, or 5-20 mM biological buffering agent at a pH of, for example, 6.0, 6.5, 6.8, 7.0, 7.2, 7.4, 7.5, 7.7, 8.0, 8.3;
up to 15 mM magnesium chloride, for example about 1-5 mM, 3, 5, 7, 10, 12, 15 mM MgCl₂;
up to 5.0% (v/v) of a non-ionic detergent, for example, 0.2-5.0%, 0.5-2.0%, 0.2, 0.5, 1.0, 1.5, 2.0, 2.5, 3.0, 4.0, 5.0% non-ionic detergent.

The buffer can further comprise a RNAse inhibitor. In some embodiments, the non-ionic detergent is selected from the group consisting of NP-40, Triton-Xn (n=100-500), a polyoxyalkylene block copolymer, a polyethylene glycol fatty acids ester, a glyceride. Additional nonionic surfactants are listed, for example, in U.S. Pat. No. 6,383,471, hereby incorporated herein by reference. In some embodiments, the biological buffering agent is selected from the group consisting of citrate, phosphate, ACES, BES, EPPS, glycine, histidine, HEPES, BIS-TRIS, TRIS and MES. Additional biological buffering agents are commercially available from, for example, Sigma-Aldrich, St. Louis, MO.

In one embodiment, the buffer comprises, consists essentially of, or consists of the following components at the following concentrations:
10 mM sodium chloride;
10 mMTRIS,pH 7.4;
3 mM magnesium chloride;
0.5% (v/v) NP-40; and
optionally, an RNAse inhibitor.

### BRIEF DESCRIPTION OF THE DRAWINGS

FIG. 1 illustrates the intact nuclei of HeLa cells subjected to Buffer E. Panel A: Phase-contrast image of intact cells not subjected to cell lysis with Buffer E. Panel B: Fluorescence image of intact cell nuclei of cells loaded with Hoechst 33258 dye and not subjected to cell lysis with Buffer E. Panel C: Phase-contrast image of intact cell nuclei of cells subjected to cell lysis with Buffer E. Panel D: Fluorescence image of intact cell nuclei of cells subjected to cell lysis with Buffer E. In cells lysed using Buffer E, the nuclei were still intact and genomic DNA was not released into the lysate. All images were taken at 40× magnification.

FIG. 2 illustrates the results of an amplification of the coagulation factor 5 gene from genomic DNA (gDNA) from fresh HeLa cells. Plots in blue: Ambion's Cells-to-cDNA™ lysis buffer. Plots in black: gDNA positive control. Plots in red: Buffer E. Amplicons from genomic DNA were not amplified from lysate prepared using Buffer E. This data is consistent with the data shown in FIG. 1.

FIG. 3 illustrates real-time reverse transcriptase polymerase chain reaction (RT-PCR) for a tubulin mRNA sequence using mRNA generated with either the Cells-to-cDNA™ kit (Ambion) and Buffer E of the present invention. Plots in teal: Ambion's Cells-to-cDNA™ lysis buffer. Plots in green: Buffer E. The Cycle threshold (Ct) values for all the dilutions for Buffer E were the same as the Ct values for the Cells-to-cDNA™ buffer. Therefore, the amount of HeLa mRNA extracted from the cells is equivalent for the two buffers. Buffer E performed as well as the commercially available buffer in the amplification of a target mRNA sequence.

### DETAILED DESCRIPTION OF THE INVENTION

Introduction

The present invention provides methods and kits for the preparation of cell lysates compatible for use in microfluidic separation techniques and which can be used directly in reverse transcriptase polymerase chain reaction (RT-PCR) amplification mixtures for the amplification of mRNA sequences without unwanted amplification of genomic DNA. The methods and kits provide for lysis of eukaryotic cells with low conductivity buffers that lyse the extracellular cell membrane without disrupting the integrity of the cell nucleus.

Definitions

The term “microfluidic” refers to devices or substrates and methods using devices or substrates that have one or more channels with at least one internal cross-sectional dimension less than 1 mm, and sometimes less than 500 μm, e.g., 0.1-500 μm.

The phrase “microfluidic separation” refers to separation of two or more molecules using a microfluidic device or substrate.

A “microfluidic substrate” is a substrate suitable for use in a microfluidic separation procedure. A microfluidic substrate can be made from any suitable material, including, for example, plastic or glass.

The terms “selective ion extraction” or “SIE” refer to a separation technique utilizing a combination of hydrodynamic flow and electrokinetic flow controls in a microfluidic device. Using selective ion extraction, mixtures of compounds or molecules sent into a T-junction on a microfluidic substrate (i.e., a microfluidic chip) can be completely separated into different channels on the basis of electrophoretic mobilities. See, for example, Kerby, et al., Anal Chem (2002) 74:5175. See also, U.S. application Ser. Nos. 10/386,900 and 10/744,915; and International Patent Publication No. WO 03/076052, the disclosures of each of which are hereby incorporated herein by reference in their entirety for all purposes.

The terms “conductivity” or “specific conductance” refer to the ability of a material or solution to conduct electrical current. Conductivity or specific conductance can be measured in units of Siemens per meter (S/m) or mhos (reciprocal ohms)/m.

Detailed Embodiments

Methods

The present methods are suitable for detecting messenger RNA (mRNA) in any eukaryotic cell, including, for example, yeast cells, insect cells, reptilian cells, amphibian cells, avian cells and mammalian cells. Cells can be prepared from tissue culture, or can be prepared directly from a tissue sample taken from a subject. For example, cells can be obtained from a biopsy tissue or from blood tissue of an individual. Cell lines for preparation from tissue culture can be obtained from, for example, the American Type Culture Collection (ATCC), Manassas, Va.

The low-conductivity lysis buffer can be prepared, if desired, as a concentrated stock solution (e.g. 5×, 10×, 20×) that can be diluted before using. In some embodiments, P-mercaptoethanol (BME), or other reducing agent, and/or a RNase inhibitor are added to the lysis buffer before contacting with the cells to be lysed.

The appropriate volume of buffer will depend on the number and size of cells to be lysed. In some embodiments, about 100 μL of lysis buffer is added to about 100,000 cells. A larger volume of lysis buffer may be used, for example, for 100,000 cells of larger size (e.g. epithelial cells, fibroblasts). A smaller volume of lysis buffer may be used, for example, for 100,000 smaller cells, (e.g., lymphocytes, including B or T cells).

The extracellular membrane of the cells is lysed by subjecting a sample of cells to a buffer of the invention, the buffer having a conductivity of about 10 mS/cm or less. The cells are incubated in the lysis buffer under conditions sufficient to effect lysis of the extracellular membrane while leaving the nuclear membrane intact. Cells can be exposed to the lysis buffer at body temperature (37° C.), room temperature (25° C.) or at a refrigerated temperature (4° C.), or other temperature, as appropriate. In one embodiment, the cells are contacted with the lysis buffer, and then placed on ice. Cell lysis usually is complete within a half hour or less, for example, within about 30, 20, 10, 5 or fewer minutes. The amount of time the cells are exposed to the lysis buffer sufficient lyse the extracellular membrane will depend on several factors, including the size of the cells, the number of cells per unit volume of lysis buffer, the temperature of incubation, etc. The cells can be further mechanically disrupted to facilitate lysis, for example, by passing through a pipettor several times, or by vortexing.

After lysis of the extracellular membrane, the intact nuclei are separated from the lysate. This can be done, for example, by centrifugation of the cell lysate. The supernatant is then separated from the pellet, containing the nuclei.

The mRNA is then removed or separated from other molecules in the supernatant, including inhibitors of reverse transcriptases and DNA polymerases, by subjecting the supernatant to a microfluidic separation procedure. The supernatant is loaded in the appropriate chambers or channels of a microfluidic substrate, for example, by injection or capillary uptake. The substrate and the cell supernatant are then subject to an electric current and/or positive or negative pressure until separation is sufficiently complete. Apparatuses and substrates for carrying out microfluidic separation procedures are commercially available, for example, from Caliper Life Sciences, Hopkinton, MA; and Agilent Technologies, Palo Alto, Calif.

In one embodiment, the microfluidic separation procedure is selective ion extraction (SIE). In SIE, fluid flow in channel segments of microfluidic substrates is driven by both positive hydrodynamic pressure and electric field and the multiple species contained in a fluid plug are separated by altering the applied pressure and electric fields in the various channel segments of the channel networks. See also, U.S. application Ser. Nos. 10/386,900 and 10/744,915; and International Patent Publication No. WO 03/076052, the disclosures of each of which are hereby incorporated herein by reference in their entirety for all purposes.

The separated mRNA-containing supernatant fraction can then be used for any purpose for which mRNA is desired. In some embodiments, the supernatant fraction containing mRNA is used directly in a reverse-transcriptase (RT) reaction or a RT-polymerase chain reaction (PCR), optionally without further processing. The supernatant fraction can be used directly in either one-step or two step RT-PCR reactions, as desired. Amplification of an RNA or DNA template using reactions is well known (see U.S. Pat. Nos. 4,683,195 and 4,683,202;PCR Protocols: A Guide to Methods and Applications (Innis et al., eds, 1990)). The reaction is preferably carried out in a thermal cycler to facilitate incubation times at desired temperatures. See, e.g., PCR PRIMER, A LABORATORY MANUAL (Dieffenbach, ed. 2003) Cold Spring Harbor Press. Exemplary PCR reaction conditions allowing for amplification of an amplicon typically comprise either two or three step cycles. Two step cycles have a denaturation step followed by a hybridization/elongation step. Three step cycles comprise a denaturation step followed by a hybridization step followed by a separate elongation step. As desired, a single or multiple mRNA sequence can be amplified. In some embodiments, a cDNA library is generated and optionally cloned into an appropriate vector for further manipulation, expression, etc.

Kits

The invention further provides kits comprising a low conductivity lysis buffer of the invention and a microfluidic substrate having one or more microfluidic channels (i.e., a microfluidic chip), as described above for the methods. The kits optionally can further comprise a control nucleic acid, for example, a control mRNA sequence. In addition, the kit can include amplification reagents, including nucleotides (e.g., A, C, G and T), a DNA polymerase, a reverse transcriptase, primers (e.g., oligo dT, random or specific) and appropriate buffers, salts and other reagents to facilitate amplification reactions. The kits can also include written instructions for the use of the kit to amplify and control for amplification of a target mRNA sequence. See, e.g., Ausubel et al., Current Protocols in Molecular Biology, 1995-2006, John Wiley & Sons, or Sambrook et al., Molecular Cloning: A Laboratory Manual, 3rd ed. 2001, Cold Spring Harbor Laboratory Press.

### EXAMPLES

### Example 1

The following example demonstrates that when “Buffer E” is used for cell lysis, genomic DNA is not released into the solution because the cell nuclei are not lysed.

Materials:

Fresh HeLa cell line grown in DMEM plus 10% fetal bovine serum (FBS), 1 mM Sodium Pyruvate and Non-essential amino acids
Phosphate-buffered saline (PBS)
Trypsin
HeLa S3 RNA (Ambion)
Human gDNA
iScript cDNA synthesis kit
iQ Sybrgreen Supermix
iQ Supermix
Primers for the amplification of the human tubulin gene from mRNA
Primers for the amplification of the coagulation factor 5 gene from genomic DNA
Recombinant RNasin Ribonuclease inhibitor (Promega Catalog number N2511)
Cells-to-cDNA™II (Ambion)
Aurum Total RNA Mini Kit
Buffer E: 10 mM NaCl, 10 mM Tris, pH 7.4, 3 mM MgCl₂, and 0.5% NP40.
   Equipment:
Vortexer
iCycler/MyCycler
Cell Culture Hood
Hemocytometer
   Procedures:
   Cell Culture:

HeLa cells were seeded in 75 cm² rectangular flasks and were allowed to grow until cells reached 90-95% confluency. Cells were then collected by trypsin treatment. After cells were counted, aliquots of approximately 100,000 cells were prepared.

Nuclei Staining:

HeLa cells were also seeded in 24-well adherent cell culture plates and were allowed to grow until cells reached 90-95% confluency. Initially, the cells in the 24-well plates were stained with the Hoechst 33258 dye to allow visual inspection of the cell nuclei under UV light using a microscope. Intact nuclei were easily identified because they emit blue fluorescent light when the dye binds double stranded DNA. Buffer E was then added to the wells of the plate. The plates were vortexed to further facilitate lysis. The lysates were viewed under the microscope to investigate if the nuclei were lysed by the buffers.

Cell Lysis

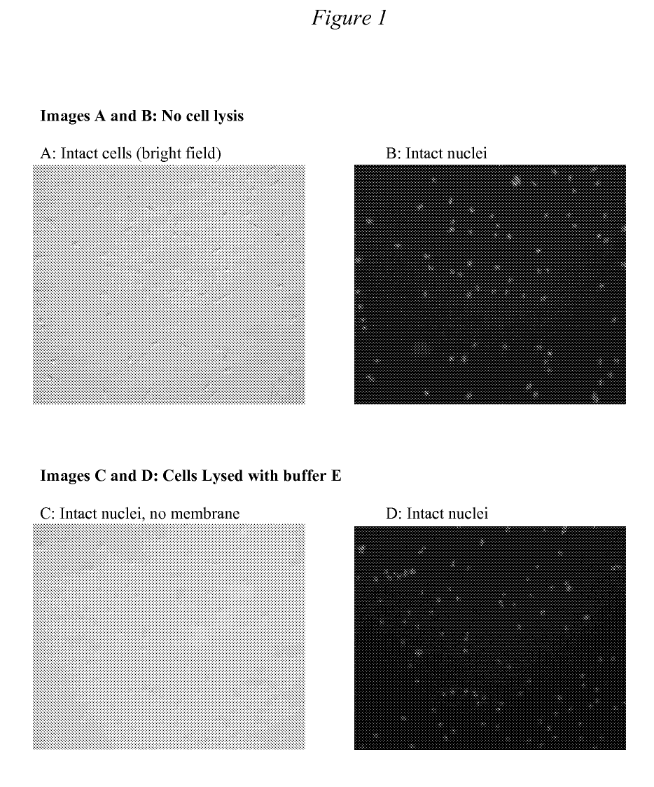

Buffer E was prepared from the 10× stock solution and 1% BME and the RNase inhibitor were added. 100 μL of each lysis buffer (Buffer E and Ambion Cells-to-cDNA™ II buffer (“C-to-C buffer”) was added to 100,000 HeLa cell aliquots. Cell lysates were passed through the pipettor several times and vortexed to further break down the cells. The Cells-to-cDNA lysate was incubated at 75° C. for 10 minutes using a thermal cycler, following the kit's instruction manual. All the cell lysates were centrifuged at 5,000×g for 10 minutes at room temperature to remove cell debris. After spinning, the supernatant from each lysate was transferred to a fresh tube and was placed on ice. Intact nuclei of cells lysed with Buffer E are shown in FIG. 1.

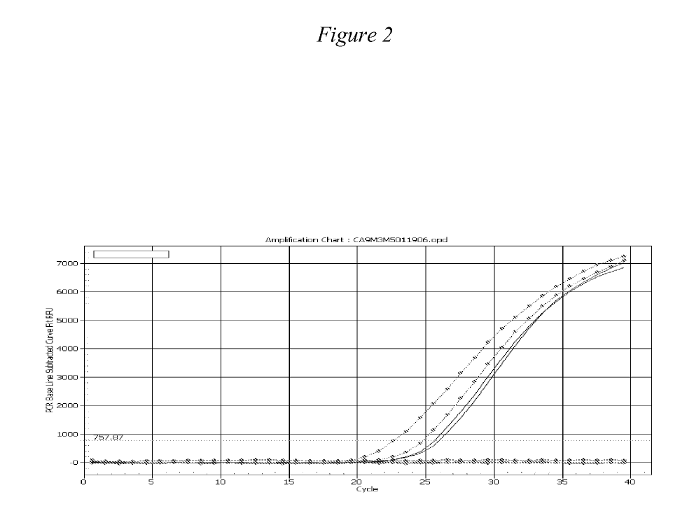

Real-time PCR for the amplification of the coagulation factor 5 gene from human gDNA was performed using 2 μL of the cell lysate for each buffer for a 50 μL PCR reaction. The reactions were performed in duplicate. The results are shown in FIG. 2.

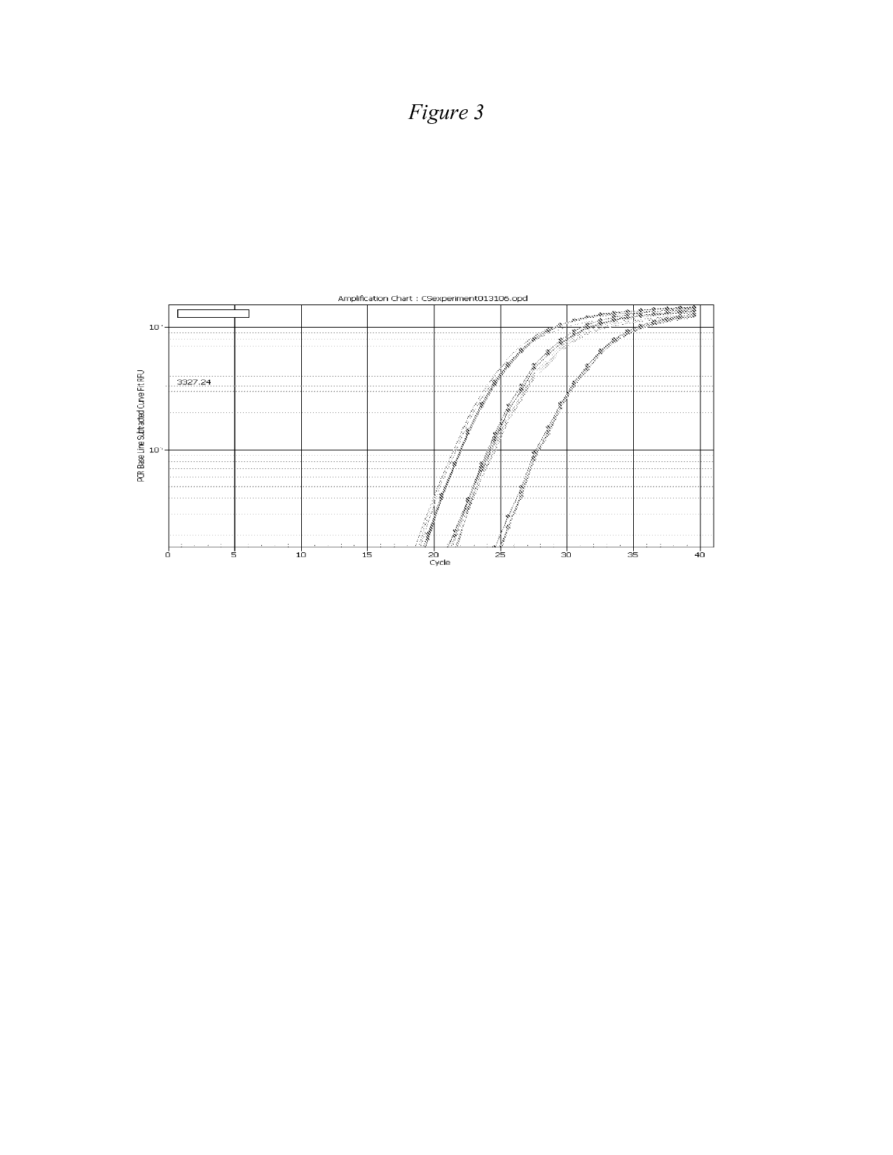

A ten-fold dilution series was prepared from the cell lysates. 2 μL from each of the dilution samples were used to prepare a 40 μL cDNA synthesis using reverse transcriptase. After cDNA synthesis, real-time PCR for tubulin gene amplification was performed in duplicate using 10 μL of cDNA for a 100 μL PCR reaction. The reactions were performed in triplicate. The results are shown in FIG. 3.

Conclusions:

Using Buffer E, we lysed HeLa cells and used an aliquot of the cell lysate directly in RT-PCR reactions. Buffer E allowed for the amplification of a target mRNA sequence equivalent to a commercially available kit, but did not disrupt the nuclei, thereby avoiding or minimizing the introduction of contaminating genomic DNA.

The above example is provided to illustrate the invention but not to limit its scope. Other variants of the invention will be readily apparent to one of ordinary skill in the art and are encompassed by the appended claims. All publications, databases, Genbank sequences, patents, and patent applications cited herein are hereby incorporated by reference.

What is claimed is:

1. A method for detecting mRNA in a cell, the method comprising
a) contacting a cell with a buffer, thereby generating a cell lysate comprising an intact cell nucleus, wherein the buffer has a conductivity of 10 mS/cm or less;
b) separating the cell nucleus from the lysate, thereby producing a supernatant containing the mRNA;
c) removing mRNA in the supernatant from inhibitors of reverse transcriptase and inhibitors of DNA polymerase via microfluidic separation; and
d) amplifying at least one mRNA sequence with reverse-transcriptase polymerase chain reaction (RT-PCR), whereby the mRNA in the cell is detected.

2. The method of claim 1, wherein the microfluidic separation comprises selective ion extraction (SIE).

3. The method of claim 1, wherein the separating step b) comprises centrifugation.

4. The method of claim 1, wherein the buffer has a conductivity of 5 mS/cm or less.

5. The method of claim 1, wherein the buffer has a conductivity of 2 mS/cm or less.

6. The method of claim 1, wherein the buffer comprises the following components at the following concentrations:
up to 50 mM sodium chloride;
up to 50 mM biological buffering agent, pH 6.0-8.3;
up to 15 mM magnesium chloride;
up to 5.0% (v/v) of a non-ionic detergent.

7. The method of claim 6, wherein the buffer further comprises a RNAse inhibitor.

8. The method of claim 6, wherein the non-ionic detergent is NP-40.

9. The method of claim 6, wherein the biological buffering agent is selected from the group consisting of citrate, phosphate, TRIS and MES.

10. The method of claim 1, wherein the buffer comprises the following components at the following concentrations:
10 mM sodium chloride;
10 mM TRIS, pH 7.4;
3 mM magnesium chloride;
0.5% (v/v) NP-40; and
an RNAse inhibitor.

11. The method of claim 1, wherein genomic DNA is not detectably amplified.

12. A kit comprising:
i) a substrate comprising microfluidic channels; and
ii) a buffer having a conductivity of 10 mS/cm or less.

13. The kit of claim 12, wherein the buffer has a conductivity of 5 mS/cm or less.

14. The kit of claim 12, wherein the buffer has a conductivity of 2 mS/cm or less.

15. The kit of claim 12, wherein the buffer comprises the following components:
up to 50 mM sodium chloride;
up to 50 mM biological buffering agent, pH 6.0-8.3;
up to 15 mM magnesium chloride;
up to 5.0% (v/v) of a non-ionic detergent.

16. The kit of claim 15, wherein the buffer further comprises a RNAse inhibitor.

17. The kit of claim 15, wherein the non-ionic detergent is NP-40.

18. The kit of claim 15, wherein the biological buffering agent is selected from the group consisting of citrate, phosphate, TRIS and MES.

19. The kit of claim 12, wherein the buffer comprises the following components:
10 mM sodium chloride;
10 mM TRIS, pH 7.4;
3 mM magnesium chloride;
0.5% (v/v) NP-40; and an RNAse inhibitor.
